(19) United States
(12) Patent Application Publication
(10) Pub. No.: US 2014/0135742 A1
(43) Pub. Date: May 15, 2014

(54) Cavitation reduction in catheter removal

(71) Applicant: John T.R. Wilson, Seattle, WA (US)

(72) Inventor: John T. R. Wilson, Seattle, WA (US)

(21) Appl. No.: 13/998,397

(22) Filed: Jan. 13, 2014

(51) Int. Cl. A61M 25/00

(52) U.S. Cl. CPC A61M 25/0043; A61M 25/0017
USPC 604/544

(57) ABSTRACT

This invention identifies cavitation as a damaging problem in catheterization occurring upon removal of the catheter from the urethra. This invention is means for reducing cavitation related damage to the urinary track upon catheter removal. This invention shows vent means to reduce cavitation damage can be plastic “trimmer lines” used in lawn trimmers also called “weed eaters”.

### BACKGROUND OF THE INVENTION

This invention relates to the human urinary track and to draining urine from the bladder by catheterizing. Such catheterizing sometimes causes bleeding. Certain urinary track bleeding related to catheterization is the problem solved by this invention. This invention is the first to identify cavitation as a damage source in catheterizing. This invention is the first to show means for reducing urinary track damage and bleeding due to cavitation in catheterizing.

Cavitation is a hydraulic term, often used in pump design and action, meaning the formation of a partial vacuum in a liquid and the subsequent collapse of the vacuum causing cavitation damage to solid surfaces. In short term applications such as liquid rocket engine firings, a cavitating venturi may be used for regulating liquid flow without concern for cavitation damage.

In human catheterization, bleeding is undesirable from any source in the urinary track. Sharp edges on the catheter can cause bleeding. Catheterizing without allowing sufficient time for healing of previous bleeding sources is undesirable.

Biopsies of bladder cells are done to identify bleeding sources. Biopsies have identified impact-damaged interior bladder cells as suspected bleeding sources.

### BRIEF SUMMARY OF THE INVENTION

The invention recognizes that damaging urinary cavitation may occur after human bladder catheterization upon removal of the catheter and describes a means to reduce or avoid that cavitation. This invention describes means to reduce or avoid cavitation at the interface between a catheter containing residual urine and a body part by venting air to the catheter tip prior to removal of the catheter. In this invention, atmospheric or outside air is admitted to the body parts interface by introducing a flexible plastic “trimmer line” into the discharge end of the catheter and by pushing the flexible plastic “trimmer line” up the catheter to the body parts interface region. The flexible plastic “trimmer line” has four longitudinal channels. This action causes residual urine in the catheter to drain down the flexible plastic “trimmer line” to the catheter discharge end. After this admitting atmospheric or outside air, no partial vacuum will be formed when the catheter is removed.

### BRIEF DESCRIPTION OF THE DRAWING

This invention is described using six Figures. The FIG. 1 and FIG. 2 depict the initial stages of catheterization. FIG. 3 and FIG. 4 depict stages of catheterization following FIG. 2 without using this invention. FIG. 5 and FIG. 6 depict stages of catheterization following FIG. 2 using this invention. The FIGS. 1 through FIG. 6 depict cross-sections through the human body parts: urethra 2, sphincter 5, valve 6, bladder 4, left-kidney ureter 7 and right-kidney ureter 8.

FIG. 1 depicts bladder 4 being drained of urine 10 through catheter 1 with the valve 6 open. The catheter 1 is shown after being inserted through urethra 2, using lubricant 3 and pushing valve 6 to open to the bladder 4.

FIG. 2 depicts sphincter 5 operated to close valve 6 to interrupt the flow path from the bladder 4 to the catheter 1 thus stopping the flow of urine 10. FIG. 2 also depicts the body parts interface region 9 between valve 6 and the catheter 1.

FIG. 3 depicts the situation following the FIG. 2 situation without using this invention. FIG. 3 depicts residual urine 10 and the formation of a partial vacuum 11 in the interface region 9 and elsewhere at the start of catheter 1 removal. The partial vacuum 11 extends from the body parts interface region 9, through the valve 6 and into the bladder 4.

FIG. 4 depicts the situation that follows FIG. 3. FIG. 4 depicts the collapse of partial vacuum 11 into pieces and the impacting of residual urine 10 onto solid surfaces of bladder 4, valve 6 and ureters 7 and 8.

FIG. 5 depicts the situation following the FIG. 2 situation using this invention. FIG. 5 also depicts the use of this invention with the introduction of vent 12 into the flow path of catheter 1 to near the tip of catheter 1 in the body parts interface region 9. The vent 12 is a flexible plastic line. FIG. 5 also depicts the sphincter 5 operated to close valve 6 to interrupt the flow path from the bladder 4 to the catheter 1 thus stopping the flow of urine. FIG. 5 also depicts the introduction of atmospheric air 13 through the vent 12.

FIG. 6 depicts the situation following the FIG. 5 situation. FIG. 6 also depicts the catheter 1 partially removed from the body parts interface region 9 with no partial vacuum formation, nor vacuum collapsing, nor residual urine 10 impacting solid surfaces of bladder 4, valve 6, urethra 2, or ureters 7 and 8.

From the situations depicted in FIG. 4 and FIG. 6, to complete a catheterization, the catheter 1 would be fully withdrawn from the urethra 2.

### DETAILED DESCRIPTION OF THE INVENTION

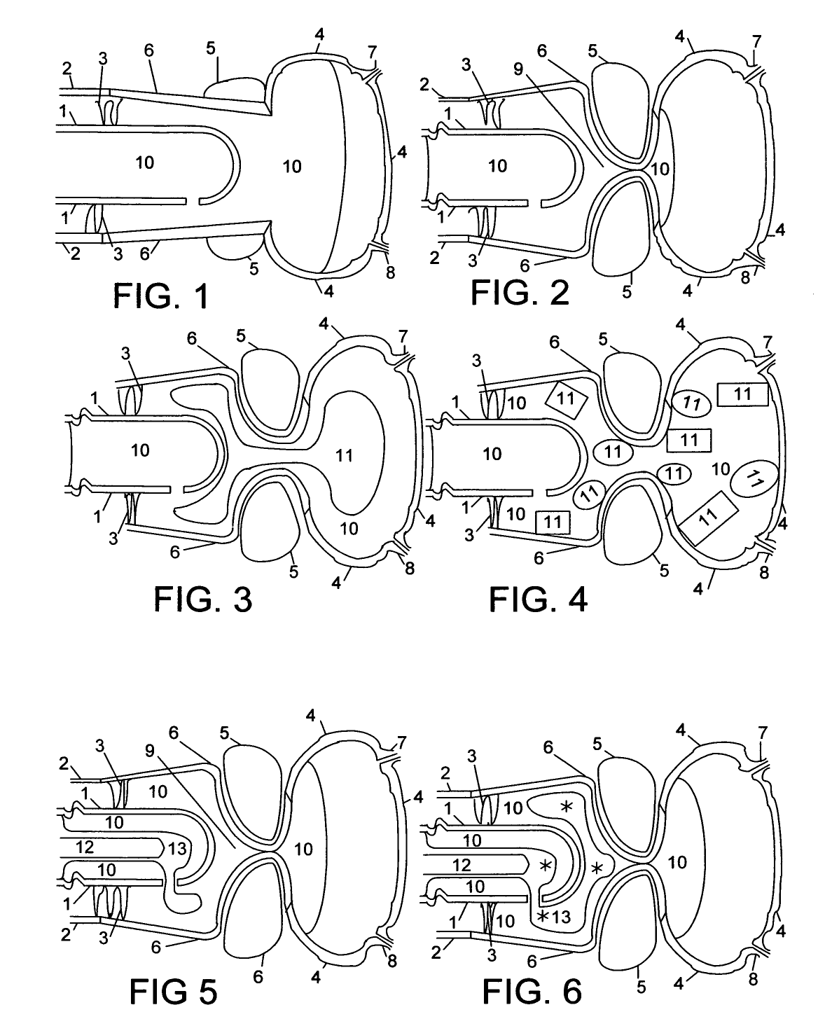

In FIG. 1 is depicted catheterization in the process of draining urine 10 from the bladder 4 into the catheter 1 via the hole in catheter 1 to the outlet of catheter 1. Catheterization starts when the catheter 1 and the mouth of urethra 2 have been coated with lubricant 3 and catheter 1 has been inserted up the urethra 2 to push open the valve 6 and start flow of urine 10.

In FIG. 2 is depicted catheterization in which the draining of urine 10 from bladder 4 has been determined to be completed and the sphincter 5 has constricted and closed valve 6. A small amount of urine 10 remains in bladder 4 and some urine 10 remains in the tip of catheter 1 and for some distance to the left in catheter 1.

The FIG. 3 depicts the situation of catheter removal without the use of subject invention means following from the FIG. 2 situation. The FIG. 3 shows catheter 1 slightly withdrawn to the left causing cavitation which is the formation of the partial vacuum 11 in body parts interface region 9 and the expansion of the partial vacuum 11 through valve 6 and into bladder 4. The urine 10 in bladder 4 is permeated by partial vacuum 11.

The FIG. 4 depicts the situation following the FIG. 3 situation. The partial vacuum 11 collapses into pieces, depicted as rectangular and spherical, next to valve 6, bladder 4 and ureters 7 and 8. The urine 10 permeates the partial vacuum 11 pieces touching valve 6, bladder 4, and ureter 7, and ureter 8 to make damaging impacts. This is called cavitation. This is what this invention prevents.

In FIG. 5 is depicted the situation following from the FIG. 2 situation, but in which the means of this invention is introduced in the form of vent 12 into the catheter 1. The vent 12 is a flexible plastic line. This vent 12 is introduced into the discharge end of catheter 1 and pushed up to the tip of catheter 1 which is in the body parts interface region 9. By this action the atmospheric air 13 is introduced into body parts interface region 9. The vent 12 used is a flexible plastic line 22 inches long and 0.065 inches diameter. The channel ends of the flexible plastic “trimmer line” are filed to look like a very small Phillips screwdriver tips to prevent gouging the inside of the catheter 1.

The FIG. 6 depicts the situation following from the FIG. 5 situation which shows the catheter 1 slightly withdrawn to the left causing a vacancy in body parts interface region 9 which is filled by atmospheric air 13; there is no partial vacuum 11 formed, and no cavitation with the associated damage.

### FUTURE CATHETERS

A catheter of the future may include an atmospheric air vent which would be controlled to connect the body-parts-to-catheter-tip interface to atmospheric air prior to removing the catheter. This control could be a motion detector for the catheter removal, a permeability or anti-permeability membrane between atmospheric air and the urine liquid or some other control.

While certain preferred embodiments of the present invention have been disclosed in detail, it is to be understood that various modifications in its structure may be adopted without departing from the spirit of the invention or the scope of the following claims.

I claim as my invention:

1. A cavitation reduction means operative between a catheter means and a body parts interface region means when the catheter means is removed from the interface region means to reduce damage to the body parts.

2. A cavitation reduction means as in claim 1 comprisng an atmospheric vent means inserted in the catheter means outlet to the interface region means.

3. A cavitation reduction means as in claim 2 wherein the atmospheric vent means comprise a flexible plastic line means.

4. A cavitation reduction means as in claim 1 wherein the catheter means comprise an atmospheric vent means.

5. A cavitation reduction means as in claim 4 wherein the catheter means comprise a liquid-soluble means blocking the vent means.

6. A cavitation reduction means as in claim 4 wherein the liquid-soluble means comprise a urine-soluble means.

7. A cavitation reduction means as in claim 3 wherein the flexible plastic line means comprise a trimmer line.

8. A cavitation reduction means as in claim 1 wherein the catheter means also comprises a controlled atmospheric vent means controlled to vent the body parts interface region means coincident with or prior to removal of the catheter means.

9. A cavitation reduction means as in claim 8 wherein the controlled atmospheric vent means comprises a membrane permeable to atmospheric air but not permeable to liquid.

10. A cavitation reduction means as in claim 8 wherein the controlled atmospheric vent means comprises a motion detector for catheter removal.
